(19) United States
(12) Patent Application Publication
(10) Pub. No.: US 2007/0167380 A1
(43) Pub. Date: Jul. 19, 2007

(54) Taste masked compositions of erythromycin a and derivatives thereof

(76) Inventors: Rahul Dabre, Magpur (IN); Vishnubhotla Nagaprasad, Hyderabad (IN); Rajiv Malik, Vienna (AT)

Correspondence Address: RANBAXY INC., 600 COLLEGE ROAD EAST, SUITE 2100, PRINCETON, NJ, 08540, US

(21) Appl. No.: 10/509,824

(22) PCT Filed: Apr. 3, 2003
(86) PCT No.: PCT/IB03/01221

§ 371 Date: Jun. 22, 2005

(30) Foreign Application Priority Data
Apr. 3, 2002 (IN) 426/DEL/2002

(51) Int. Cl. A61K 31/7048; A61K 31/734

(52) U.S. Cl. 514/29; 514/54

(57) ABSTRACT

A pharmaceutical composition includes erythromycin A or a derivative thereof and alginic acid. The alginic acid provides taste masking of the erythromycin A or derivative. The erythromycin A derivative may be clarithromycin and the alginic acid may be one or both of alginic acid and its salt. The salt may be one or more of sodium alginate and calcium alginate. The pharmaceutical composition may further include one or more of a binder, a disintegrant, a flavoring agent, and a coating. The pharmaceutical composition also may include one or more active ingredients, including omeprazole, metronidazole, amoxicillin, rifampicin, lansoprazole, ciprofloxacin, ethambutol, and ritonavir. The erythromycin A or a derivative thereof and the one or more active ingredients may be combined in a single pharmaceutical composition.

### TECHNICAL FIELD OF THE INVENTION

The field of the invention generally relates to taste masking of erythromycin A and derivatives using alginic acid.

### BACKGROUND OF THE INVENTION

Erythromycin and its derivatives are extremely bitter drugs, which when dissolved even in trace quantities in a liquid dosage form, are often perceived to be unpalatable. They are, however, also the drugs of choice for the treatment of common pediatric infections of the middle ear and the upper respiratory tract as well as certain forms of pneumonia which afflict the elderly. Administration of such drugs to children and the elderly poses a challenge as these individuals experience difficulty in swallowing solid oral dosage forms. For these patients, drugs typically are provided in liquid forms, such as solutions, emulsions and suspensions, which usually permit perceptible exposure of the active drug ingredient to the taste bud.

There is a need to mask the taste of such drugs in order to ensure patient compliance during therapy. Conventional taste masking techniques, such as the use of sweeteners, amino acids and flavoring agents often are unsuccessful in masking the taste of highly bitter drugs and, consequently, other techniques need to be exploited for effectively masking the taste of these drugs.

One such technique involves the use of cation exchange resins to adsorb amine drugs for taste masking and sustained release. It, however, has limited applicability and is not capable of masking the taste of highly bitter drugs.

Coating bitter drugs is another method which has been reported as being successful for taste masking of some drugs. Unfortunately, this technique is usually effective only for masking the taste of moderately bitter drugs where the coated particles are formulated as aqueous formulations just before administration or are formulated in a non-aqueous medium. This technology, however, has its limitations—it is technology-intensive and the coated granules are easily ruptured by chewing and compression.

Lipid based microencapsulation is another technique used for masking the taste of drugs. This technique requires highly sophisticated hot melt granulation for producing free particles, may have adverse effects on heat sensitive molecules, and may adversely restrict drug release characteristics.

U.S. Pat. No. 4,808,411 describes taste-masked compositions that include 95% of erythromycin or a derivative thereof and about 5 to about 75% of a carbomer. The drug and carbomer are believed to be held together by the ionic interactions between the amine group of the erythromycin compound and the carbonyl group of the carbomer and gel properties of the carbomer. These complexes typically are prepared by dissolving the drug in a mixture of acetone and alcohol and adding carbomer in acetone or an acetone/alcohol mixture. Utilization of these processes on an industrial scale presents a number of problems, including employee safety, emission of solvent vapors to the environment, and cost.

U.S. Pat. No. 5,919,489 describes an aqueous granulation process for overcoming the limitations of U.S. Pat. No. 4,808,411. The aqueous granulation process involves the steps of mixing a macrolide antibiotic and a carbomer in a weight ratio of between about 1:10 and about 5:2, wetting the mixture with an aqueous solvent; blending the mixture for a time sufficient to allow formation of macrolide antibiotic-carbomer granules, and drying the antibiotic-carbomer granules. The blending is accomplished in a vessel having a head space which is maintained at a temperature from about 0° to about 70° C. Like U.S. Pat. No. 4,808,411, this patent also uses a carbomer for the taste masking of clarithromycin granules.

### SUMMARY OF THE INVENTION

In one general aspect, there is provided a pharmaceutical composition which includes erythromycin A or a derivative thereof and alginic acid.

Embodiments of the pharmaceutical composition may include one or more of the following features. For example, the erythromycin A derivative may be clarithromycin. The alginic acid may be one or both of alginic acid and its salt. The salt may be one or more of sodium alginate and calcium alginate.

The erythromycin A or derivative thereof and alginic acid may be present in a ratio of approximately 2.5:1 to approximately 50:1. The particle size of erythromycin A or a derivative thereof may be less than approximately 50 microns. The erythromycin A or a derivative thereof and alginic acid may be in the form of granules, and the granules may further include pharmaceutically acceptable excipients.

The erythromycin A or a derivative thereof, alginic acid, and/or pharmaceutical excipients may surround a core.

The pharmaceutical composition may further include one or more of a binder, a disintegrant, a flavoring agent, and a coating. The pharmaceutical composition may further include one or more active ingredients that include one or more of omeprazole, metronidazole, amoxicillin, rifampicin, lansoprazole, ciprofloxacin, ethambutol, and ritonavir. The erythromycin A or a derivative thereof and the one or more active ingredients may be combined in a single pharmaceutical composition.

In another general aspect, there is provided a process for preparing a pharmaceutical composition of erythromycin A or derivative thereof which includes mixing erythromycin A or a derivative thereof and alginic acid to form a mixture.

Embodiments of the process may include one or more of the following features. For example, the process may further include granulating the mixture with an aqueous solvent, or dispersing the mixture in an aqueous solvent and layering onto one or more inert cores. The process may further include coating with a coating material.

The inert core may include one or more of microcrystalline cellulose, starch, sugar or lactose. The inert core may have a particle size of between approximately 50 microns and approximately 1000 microns and, more particularly, between approximately 100 microns and approximately 350 microns.

The process may further include mixing one or more pharmaceutically acceptable excipients with the erythromycin A or derivative and alginic acid. The pharmaceutically acceptable excipient may be one or more of a binder, a disintegrant, and a flavoring agent. The binder may be one or more of hydroxypropyl methylcellulose, hydroxypropyl cellulose, polyvinylpyrrolidone, pregelatinised starch, gelatin, and sucrose. The disintegrant may be one or more of croscarmellose sodium, sodium starch glycolate, cross-linked polyvinyl pyrrolidone, sodium carboxymethylcellulose, and starch.

The pharmaceutical composition may be formulated as a dry syrup, suspension, or conventional chewable, dispersible tablet. The erythromycin derivative may be clarithromycin.

In another general aspect, there is provided a method of treating a bacterial infection in a mammal in need of treatment which includes administering a pharmaceutical composition that includes erythromycin A or a derivative thereof and alginic acid.

Embodiments of the method of treatment may include one or more of the following features. For example, the erythromycin derivative may be clarithromycin. The alginic acid may be one or both of alginic acid and its salt and the salt may be one or more of sodium alginate and calcium alginate.

The erythromycin A or derivative thereof and alginic acid may be present in a ratio of approximately 2.5:1 to approximately 50:1. The particle size of erythromycin A or a derivative thereof may be less than approximately 50 microns.

The method may further include administering one or more of omeprazole, metronidazole, amoxicillin, rifampicin, lansoprazole, ciprofloxacin, ethambutol, and ritonavir with the erythromycin A or derivative thereof.

In another general aspect, a method of masking the taste of erythromycin A or a derivative thereof in a pharmaceutical composition includes mixing the erythromycin A or derivative thereof with alginic acid.

Embodiments of the taste masking method may include any of the features described above. For example, the erythromycin derivative may be clarithromycin. The erythromycin A or a derivative thereof may be mixed with the alginic acid in a ratio of between approximately 2.5:1 to approximately 50:1.

The details of one or more embodiments of the invention are set forth in the description below. Other features, objects, and advantages of the invention will be apparent from the description and claims.

### DETAILED DESCRIPTION OF THE INVENTION

We have now discovered that erythromycin A or a derivative thereof, such as clarithromycin, when blended with alginic acid results in a composition which has improved palatability because the alginic acid is effective in masking the bitter taste of the active ingredient. Compared to some conventional formulations, a solid preparation of erythromycin A or a derivative thereof blended with alginic acid is characterized by a significant reduction of the bitter taste of the active ingredient. According to one embodiment, erythromycin A or a derivative thereof and alginic acid are prepared and administered in a drug to polymer ratio of approximately 2.5:1 to approximately 50:1. More particularly, this ratio may be between 10:1 to 30:1. Alginic acid may be added as alginic acid or any of its salts, including sodium alginate, calcium alginate and the like.

In general, the process for preparing taste-masked granules of erythromycin A or a derivative thereof includes the steps of mixing erythromycin A or a derivative thereof, alginic acid, and other pharmaceutically acceptable excipients, and either granulating the mixture in an aqueous solvent/media or dispersing the mixture in an aqueous solvent with subsequent layering on inert cores, such as nonpareil seeds, microcrystalline cellulose spheres etc. In the latter process, the drug-polymer (i.e., erythromycin A or derivative and alginic acid) mixture, together with the other pharmaceutically acceptable excipients, is loaded onto the inert core using a fluid bed processor. The granules obtained through either process are dried to a loss on drying of, for example, not more than approximately 4.0% at 105° C. in, for example, a fluid bed dryer.

One erythromycin derivative that may be used in accordance with the present invention is clarithromycin. Clarithromycin is known as useful agent in treating bacterial infections. For improved results, the clarithromycin should be micronized, or otherwise have its particle size reduced, to have a particle size less than approximately 50 microns.

The above inert cores may be made up of microcrystalline cellulose, starch, sugar, or lactose. As a particular example, the inert cores may be made from the microcrystalline cellulose that is sold under the trade name of Celphere™ seeds. The particle size of the inert cores used in the taste-masked composition is important to providing the taste masking and palatability of the composition. For example, if the particle size is too small, there are too many fines and hence ineffective masking of the taste. On the other hand, if the particle size is large, the formulation is overly gritty. The particle size of the inert cores therefore is kept in the range of from approximately 50 microns to approximately 1000 microns and, in particular, between approximately 100 microns and approximately 350 microns.

As described above, the granules may further include pharmaceutically acceptable excipients, such as binders and disintegrants. Binders are added to add cohesiveness to the coating composition. Various binders of differing adhesive strength are known in the art and may be selected from amongst those commonly known in the art, including hydroxypropyl methylcellulose, hydroxypropyl cellulose, polyvinylpyrrolidone, pregelatinized starch, gelatin, sucrose, and the like. The binder is present at a drug to binder ratio of from about 4:1 to about 1:4.

If desired to release all or a majority of the drug rapidly upon ingestion, it may be necessary to add disintegrants to the formulation. These disintegrants may be selected from amongst those commonly known in the art such as croscarmellose sodium, cross-linked polyvinylpyrrolidone, sodium starch glycolate, sodium carboxymethylcellulose, starch and the like.

The examples given herein further illustrate the effectiveness of our formulation in achieving both taste masking and optimal dissolution of the drug from the matrix.

As presented below for Examples 1-4 in Table 1, hydroxypropyl cellulose and hydroxypropyl methyl cellulose were dispersed in water together with croscarmellose sodium and, optionally, alginic acid (Examples 1-3). Clarithromycin and, optionally, Tween 80 (Example 2) were added to the dispersion. This dispersion was then coated on microcrystalline cellulose beads in a fluid bed processor to achieve a weight build up of approximately 140%. The granules were dried in a fluid bed dryer. The granules were optionally then mixed with iron oxide yellow (Example 2).

In Examples 1-4, the effect of taste-masking of clarithromycin with different amounts of alginic acid was studied. The granules obtained when no alginic acid was used in the composition (Example 4) were highly bitter. However, the addition of even small amounts of alginic acid (Examples 1 to 3) was enough to perceptibly reduce the bitterness of the formulation. All of the formulations described above released more than 70% of the drug at pH 6.8 at 50 rpm within 45 minutes.

TABLE 1
Effect on Taste Masking Achieved by Varying the Amount of
Alginic Acid Present Using a Dispersion Production Process

| | Amount (mg) | | | |
|---|---|---|---|---|
| Ingredients | Ex. 1 | Ex. 2 | Ex. 3 | Ex. 4 |
| Microcrystalline cellulose beads | 250.0 | 150.0 | 250.0 | 250.0 |
| Clarithromycin | 250.0 | 150.0 | 250.0 | 250.0 |
| Alginic acid | 12.5 | 30 | 25.0 | — |
| Hydroxypropyl methylcellulose | 61.5 | — | 61.5 | 61.5 |
| Hydroxypropyl cellulose | 6.15 | — | 6.15 | 6.15 |
| Tween 80 | — | 0.3 | — | — |
| Water | qs | 1300.0 | qs | qs |
| Iron Oxide Yellow | — | 1.0 | — | — |
| Croscarmellose sodium | 20 | — | 20 | 20 |

Using the granulation process described above, the drug was granulated with alginic acid in the quantities described in Table 2. In Examples 5 and 6, clarithromycin, croscarmellose sodium, sucrose, and, optionally, hydroxypropyl methylcellulose were sifted and granulated with a solution of sodium alginate in water. The taste masked granules obtained were dried in a fluid bed dryer. The granules of Examples 5 and 6 above were sufficiently taste masked for formulating into a suitable oral dosage form.

TABLE 2
Effect on Taste Masking Achieved by Varying the Amount of
Alginic Acid Present Using a Dispersion Production Process

Amount (mg)

| Ingredient | Example 5 | Example 6 |
|---|---|---|
| Clarithromycin | 250 | 250 |
| Sodium alginate | 125 | 62.5 |
| Hydroxypropyl | — | 62.5 |
| methylcellulose E5 | | |
| Croscarmellose Sodium | 15 | 15 |
| Sucrose | 50 | 50 |

To further reduce the dissolution or release of the active drug in the mouth where it can be perceived by the taste buds, the granules of Examples 1-6 may be coated with a polymer. A variety of polymeric materials can be employed to achieve this coating. Non-limiting examples of such polymeric materials include ethyl cellulose, hydroxypropyl cellulose, hydroxypropyl methylcellulose phthalate, shellac, and methacrylate polymers, such as those sold under the tradename Eudragit™ E100, S100 and L-100 available from Rohm and Haas Company. A particularly suitable polymer is hydroxypropyl methylcellulose phthalate. The use of pH sensitive coatings, such as Eudragit™, have particular advantage for use with acid labile drugs, such as clarithromycin, because the pH sensitive coating material is insoluble in acid or water while dissolving in neutral buffer above pH 5 or 6. This permits the formulator to prepare a suspension of coated clarithromycin-polymer granules that remain intact in the stomach yet release the antibiotic in the intestine. This controlled release advantageously protects the drug from the hostile, acidic environment of the stomach while releasing the drug rapidly at the higher pH of the intestinal tract.

Further, the taste masked granules of Examples 1-6, with or without the polymer coating, may be mixed with flavoring agents, such as natural or artificial flavors, citric and tartaric acids, sweeteners, such as saccharin and aspartame, and with other pharmaceutically acceptable excipients, such as pH modifiers, thickeners, etc. to be formulated as a conventional, chewable, dispersible tablet, dry syrup, suspension, sachet, or any other suitable oral dosage form.

While several particular forms of the invention have been illustrated and described, it will be apparent that various modifications and combinations of the invention detailed in the text can be made without departing from the spirit and scope of the invention. For example, the erythromycin A or derivative thereof may be administered with (e.g., as a single pharmaceutical combination composition, simultaneously, or within a short time) other drugs and drug products to treat conditions that may be related to or occur concurrently with a condition that involves the treatment of a bacterial infection using erythromycin A or a derivative, such as clarithromycin. Such drugs that may be co-administered with the micronized clarithromycin generally include one or more of omeprazole, metronidazole, amoxicillin, rifampicin, lansoprazole, ciprofloxacin, ethambutol, and ritonavir. For example, the combinations may include a single pharmaceutical composition or joint administration of: (1) omeprazole, metronidazole, and clarithromycin; (2) omeprazole, amoxicillin, and clarithromycin; (3) rifampicin and clarithromycin; (4) lansoprazole and clarithromycin; (5) ciprofloxacin and clarithromycin; (6) lansoprazole, amoxicillin, and clarithromycin; and (7) ethambutol, ritonavir, and clarithromycin.

Further, it is contemplated that any single feature or any combination of optional features of the inventive variations described herein may be specifically excluded from the claimed invention and be so described as a negative limitation. Accordingly, it is not intended that the invention be limited, except as by the appended claims.

We claim:

1. A pharmaceutical composition comprising erythromycin A or a derivative thereof and alginic acid.

2. The pharmaceutical composition of claim 1, wherein the erythromycin A derivative comprises clarithromycin.

3. The pharmaceutical composition of claim 1, wherein the alginic acid comprises one or both of alginic acid and its salt.

4. The pharmaceutical composition of claim 3, wherein the salt comprises one or more of sodium alginate and calcium alginate.

5. The pharmaceutical composition of claim 1, wherein the erythromycin A or derivative thereof and alginic acid are present in a ratio of approximately 2.5:1 to approximately 50:1.

6. The pharmaceutical composition of claim 1, wherein the particle size of erythromycin A or a derivative thereof is less than approximately 50 microns.

7. The pharmaceutical composition of claim 1, wherein the erythromycin A or a derivative thereof and alginic acid comprise granules.

8. The pharmaceutical composition of claim 7, wherein the granules further comprise pharmaceutically acceptable excipients.

9. The pharmaceutical composition of claim 1, wherein the erythromycin A or a derivative thereof and alginic acid surround a core.

10. The pharmaceutical composition of claim 9, further comprising pharmaceutically acceptable excipients surrounding the core.

11. The pharmaceutical composition of claim 1, further comprising one or more of a binder, a disintegrant, a flavoring agent, and a coating.

12. The pharmaceutical composition of claim 1, further comprising one or more active ingredients, wherein the active ingredients comprise one or more of omeprazole, metronidazole, amoxicillin, rifampicin, lansoprazole, ciprofloxacin, ethambutol, and ritonavir.

13. The pharmaceutical composition of claim 12, wherein the erythromycin A or a derivative thereof and the one or more active ingredients are combined in a single pharmaceutical composition.

14. A process for preparing a pharmaceutical composition of erythromycin A or derivative thereof, the process comprising:
mixing erythromycin A or a derivative thereof and alginic acid to form a mixture.

15. The process of claim 14, further comprising granulating the mixture with an aqueous solvent.

16. The process of claim 14, further comprising dispersing the mixture in an aqueous solvent and layering onto one or more inert cores.

17. The process of claim 14, further comprising coating with a coating material.

18. The process of claim 16, wherein the inert core comprises one or more of microcrystalline cellulose, starch, sugar or lactose.

19. The process of claim 18, wherein the inert core comprises microcrystalline cellulose.

20. The process of claim 18, wherein the inert core has a particle size of between approximately 50 microns and approximately 1000 microns.

21. The process of claim 18, wherein the inert core has a particle size of between approximately 100 microns and approximately 350 microns.

22. The process of claim 14, further comprising mixing one or more pharmaceutically acceptable excipients with the erythromycin A or derivative and alginic acid.

23. The process of claim 22, wherein the pharmaceutically acceptable excipient comprises one or more of a binder, a disintegrant, and a flavoring agent.

24. The process of claim 23, wherein the binder comprises one or more of hydroxypropyl methylcellulose, hydroxypropyl cellulose, polyvinylpyrrolidone, pregelatinised starch, gelatin, and sucrose.

25. The process of claim 23, wherein the disintegrant comprises one or more of croscarmellose sodium, sodium starch glycolate, cross-linked polyvinyl pyrrolidone, sodium carboxymethylcellulose, and starch.

26. The process of claim 14 wherein the pharmaceutical composition is formulated as a dry syrup, suspension, or chewable, dispersible tablet.

27. The process of claim 14, wherein the erythromycin derivative comprises clarithromycin.

28. A method of treating a bacterial infection in a mammal in need of treatment, the method comprising administering a pharmaceutical composition comprising erythromycin A or a derivative thereof and alginic acid.

29. The method of claim 28, wherein the erythromycin derivative comprises clarithromycin.

30. The method of claim 28, wherein the alginic acid comprises one or both of alginic acid and its salt.

31. The method of claim 30, wherein the salt comprises one or more of sodium alginate and calcium alginate.

32. The method of claim 28, wherein the erythromycin A or derivative thereof and alginic acid are present in a ratio of approximately 2.5:1 to approximately 50:1.

33. The method of claim 28, wherein the particle size of erythromycin A or a derivative thereof is less than approximately 50 microns.

34. The method of claim 28, further comprising administering one or more of omeprazole, metronidazole, amoxicillin, rifampicin, lansoprazole, ciprofloxacin, ethambutol, and ritonavir with the erythromycin A or derivative thereof.

35. A method of masking the taste of erythromycin A or a derivative thereof in a pharmaceutical composition, the method comprising mixing the erythromycin A or derivative thereof with alginic acid.

36. The method of claim 35, wherein the erythromycin derivative comprises clarithromycin.

37. The method of claim 35, wherein the erythromycin A or a derivative thereof is mixed with the alginic acid in a ratio of between approximately 2.5:1 to approximately 50:1.
